(19) United States
(12) Patent Application Publication
(10) Pub. No.: US 2024/0293808 A1
(43) Pub. Date: Sep. 5, 2024

(54) APPARATUS FOR TESTING A LIQUID SPECIMEN

(71) Applicant: CLINICAL DESIGN TECHNOLOGIES LIMITED, Devon (GB)

(72) Inventor: Oliver BLACKWELL, Devon (GB)

(21) Appl. No.: 18/268,197

(22) PCT Filed: Dec. 10, 2021
(86) PCT No.: PCT/GB2021/000141

§ 371 Date: Jun. 16, 2023

(30) Foreign Application Priority Data
Dec. 17, 2020 (GB) 2020009.3

(51) Int. Cl. B01L 3/00

(52) U.S. Cl. CPC B01L 3/502; B01L 2200/02; B01L 2300/041; B01L 2300/0609; B01L 2400/06

(57) ABSTRACT

Apparatus (2) for testing a liquid specimen (4), which apparatus (2) comprises; (i) a test container (6); (if) a plurality of response portions (8) which are positioned in the test container (6) and which enable the testing of the liquid specimen consequent upon contact with the liquid specimen (4); and (Hi) mover means (10) for moving the response portions (8) out of the test container (6) for contact with the liquid specimen (4), and for moving the response portions (8) back into the test container (6) after the contact with the liquid specimen (4) and for the testing of the liquid specimen (4).

This invention relates to apparatus for testing a liquid specimen, for example a bodily fluid such as urine.

Urine is currently tested manually using a dip stick process. More specifically, a strip containing response portions which are responsive to the urine is dipped into the urine and is then removed. Response portions on the strip have to be fully submerged in the urine. The strip is then removed from the urine and an operator is required to remove excess urine from the strip by scraping the strip on the side of a collection container containing the urine. Any remaining unwanted excess urine on the strip is then removed by blotting the strip onto a piece of paper towel. The entire manual procedure is unpleasant and unhygienic. Other types of liquid specimens may similarly be tested, or they may be tested in other ways which are also not as pleasant and hygienic as would be desired.

It is an aim of the present invention to reduce the above mentioned problems.

Accordingly, in one non-limiting embodiment of the present invention there is provided apparatus for testing a liquid specimen, which apparatus comprises:
(i) a test container;
(ii) a plurality of response portions which are positioned in the test container and which enable the testing of the liquid specimen consequent upon contact with the liquid specimen; and
(iii) mover means for moving the response portions out of the test container for contact with the liquid specimen, and for moving the response portions back into the test container after the contact with the liquid specimen and for the testing of the liquid specimen.

The apparatus of the present invention is advantageous in that the mover means enables the response portions to start and finish in the test container. This helps to avoid the above mentioned problems associated with the known manual testing of liquid specimens.

The apparatus of the present invention may be one in which the contact with the liquid specimen comprises dipping the response portions into the liquid specimen, and in which the moving of the response portions back into the test container comprises removing the response portions from the liquid specimen. The apparatus of the present invention may operate other than by way of the dipping. Thus, for example, the apparatus may operate with the liquid specimen being poured or otherwise brought into contact with the response portions.

The apparatus may include a collection container for the liquid specimen, and in which the mover means moves the response portions into the collection container for the contact with the liquid specimen, and in which the mover means moves the response portions out of the collection container for moving the response portions back into the test container. The collection container provides a convenient means for containing the liquid specimen. However, if desired, the apparatus of the present invention may be used without the collection container.

The apparatus may be one in which the collection container comprises a body and a lid, and in which the mover means moves the response portions into and out of the collection container through the lid. Other arrangements may be employed so that, for example, the mover means may move the response portions into and out of the collection container through a side of the body of the collection container.

The response portions may move into and out of the collection container through valve means. In this case, the apparatus may be one in which the valve means comprises a valve orifice, and in which the mover means opens the valve orifice such that the response portions do not touch the valve means. Thus the response portions may be such that they do not touch the valve means by, for example, touching the sides of the valve means defining the orifice. Other types of valve means may be employed.

The response portions may be connected to the mover means. Other arrangements may be employed so that, for example, the response portions may not be connected to the mover means.

The mover means may be a linear acting mover means. The linear acting mover means may be a plunger linear acting mover means. Other types of mover means may be employed so that, for example, the mover means may be a rotary acting mover means.

The plunger linear acting mover means may comprise an elongate body and a plunger for moving to and fro in the elongate body, and in which the response portions are connected to the plunger. The response portions may extend longitudinally. The response portions may extend in other shapes if desired.

The apparatus may include remover means for removing unwanted liquid specimen from the response portions after the contact of the response portions with the liquid specimen.

The remover means may be a scraping remover means which operates to scrape the unwanted liquid specimen from the response portions after the contact of the response portions with the liquid specimen. Alternatively, the remover means may be a sponge remover means. The sponge remover means may operate by absorption. Other types of remover means may be employed so that, for example, the remover means may be a squeezing remover means.

When the remover means is a scraping remover means, then the apparatus may be one in which the scraping remover means comprises a slot for receiving the response portions, and in which at least a part of the slot is defined by a wall which scrapes against at least a part of the response portions. The wall can be of any required shape and/or material for making required contact with all or part of the response portions in order to remove the unwanted liquid specimen.

The apparatus may be one in which at least a part of the test container is transparent in order to allow reading of the response portion in the test container after the response portions have been moved into contact with the liquid specimen and moved back into the test container. All or part of the test container may be transparent. When only a part of the test container is transparent, then the transparent part of the test container may be in the form of a viewing window. The viewing window may be of the same shape as the collective shape defined by all of the response portions. Thus, for example, if all of the response portions form a strip, then the viewing window may be a viewing strip. Similarly, if the collective shape defined by all of the response portions is a circle, then the viewing window may be in the form of a circle.

An embodiment of the invention will now be described solely by way of example and with reference to the accompanying drawings in which:

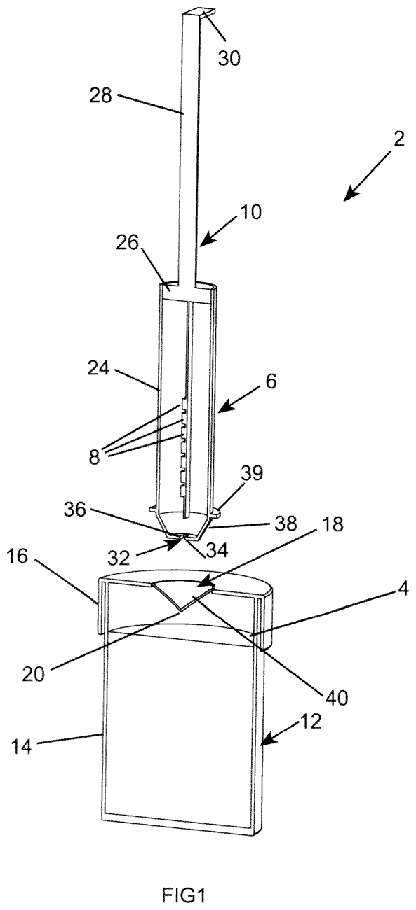

FIG. 1 is a section through apparatus for testing a liquid specimen, with the apparatus being shown in a first condition;

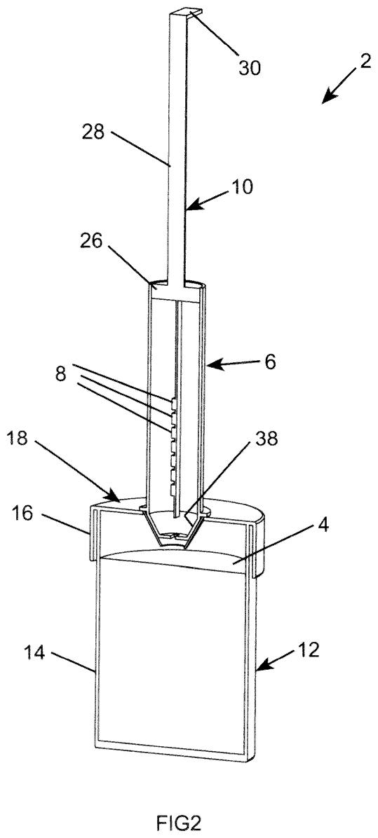

FIG. 2 is a section like FIG. 1 but with the apparatus being in a second condition;

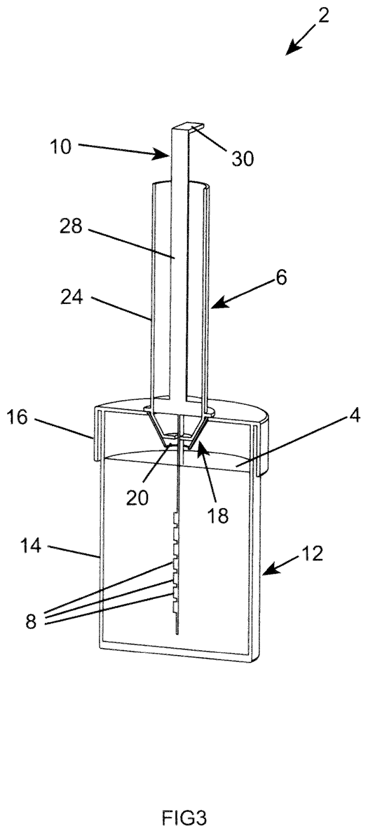

FIG. 3 is a section like FIG. 1 but with the apparatus being in a third condition;

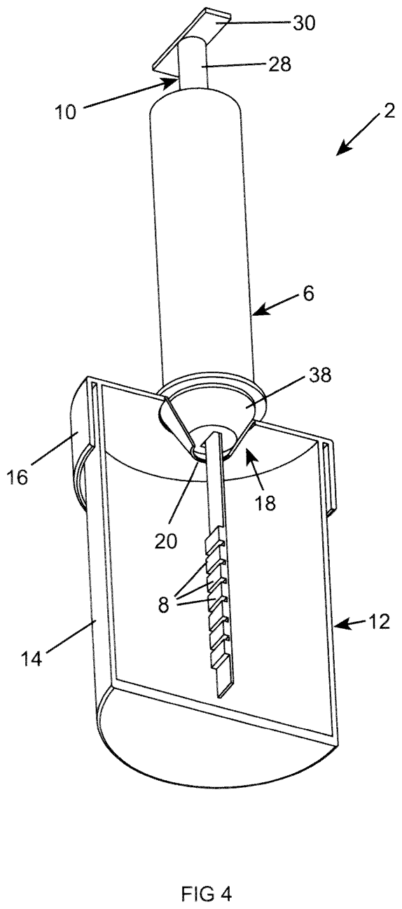

FIG. 4 is a part sectional view from underneath of the apparatus in the third condition shown in FIG. 3;

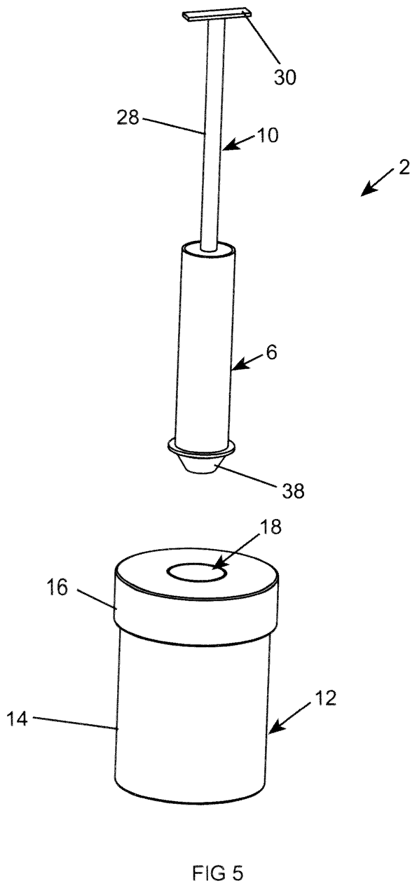

FIG. 5 is a perspective view of the apparatus as shown in FIG. 1;

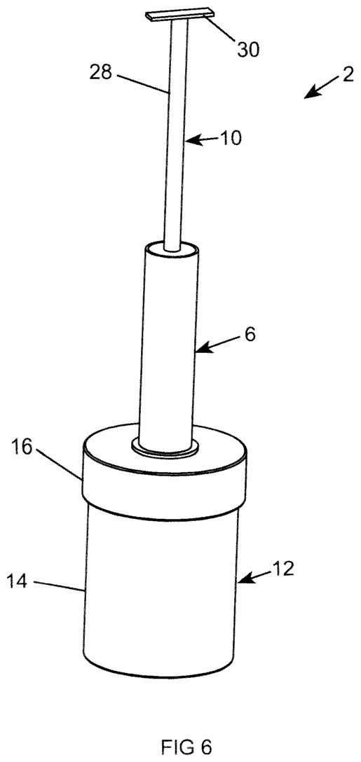

FIG. 6 is a perspective view of the apparatus as shown in FIG. 2; and

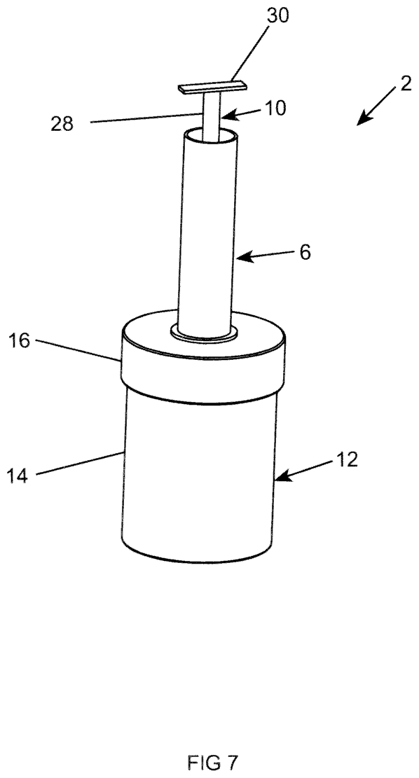

FIG. 7 is a perspective view of the apparatus as shown in FIG. 3.

Referring to the drawings, there is shown apparatus 2 for testing a liquid specimen 4. The apparatus 2 comprises a test container 6. A plurality of response portions 8 are positioned in the test container 6. The response portions 8 enable the testing of the liquid specimen 4 consequent upon contact with the liquid specimen 4.

The apparatus 2 further comprises mover means 10. The mover means 10 is for moving the response portions 8 out of the test container 6 for contact with the liquid specimen 4. The mover means 10 is also for moving the response portions 8 back into the test container 6 after the contact with the liquid specimen 4 and for the testing of the liquid specimen 4. The contact with the liquid specimen 4 comprises dipping the response portions 8 into the liquid specimen 4. The moving of the response portions 8 back into the test container 6 comprises removing the response portions 8 from the liquid specimen 4.

The apparatus 2 includes a collection container 12 for the liquid specimen 4. The mover means 10 moves the response portions 8 into the collection container 12 for the contact with the liquid specimen 4. The mover means 10 moves the response portions 8 out of the collection container 12 for the moving of the response portions 8 back into the test container 6.

The collection container 12 comprises a body 14 and a lid 16. The mover means 10 moves the response portions 8 into and out of the collection container 12 through the lid 16.

The response portions 8 moving into and out of the collection container 12 through valve means 18. The valve means 18 comprises a valve orifice 20. The test container 6 opens the valve orifice 20 such that the response portions 8 do not touch the valve means 18. More specifically, the response portions 8 do not touch the side of the valve means 18 defining the valve orifice 20. The test container 6 terminates in a frusto-conical portion 38 which sits in a V-formation forming part of the valve means 18. As can be appreciated from a comparison of FIGS. 1 and 2, the pushing of the frusto-conical portion 38 into the V-formation 40 causes the valve orifice 20 to open up. This opening is such that the response portions 8 do not touch the valve means 18. The frusto-conical portion 38 has a flange 39 which limits the distance that the test container 6 can penetrate the valve orifice 20. This thereby avoids possible stressing of the valve means 18.

The frusto-conical portion 38 has an angle of inclination which is steeper than the receiving V-formation 40. Thus, when the frusto-conical portion 38 is pushed into the V-formation 40, the frusto-conical portion 38 causes the valve orifice 20 to open sufficiently wide that there is a space around the response portions 8 when the mover means 10 moves the response portions 8 into and out of the collection container 12. This prevents excess liquid specimen 4 being transferred to the outside surfaces of the V-formation 40 by the response portions 8.

The response portions 8 are connected to the mover means 10. The mover means 10 is a linear acting mover means 10. More specifically, the linear acting mover means 10 is a plunger linear acting mover means 10. The plunger linear acting mover means 10 comprises an elongate body 24 and a plunger 26 for moving to and fro in the elongate body 24. The elongate body 24 forms part of the test container 6. The response portions 8 extend longitudinally and they are connected to the plunger 26. The plunger 26 is connected to a handle 28 which terminates in a hand grip portion 30. The handle 28 with its hand grip portion 30 enables the plunger 26 to be pushed downwardly along the elongate body 24 from the position shown in FIGS. 1 and 2 to the position shown in FIGS. 3 and 4. The handle 28 and its hand grip portion 30 then enable the plunger 26 to be pulled back along the elongate body 24 to the position shown in FIGS. 1 and 2.

The apparatus 2 comprises remover means 32 for removing unwanted liquid specimen from the response portions 8 after the contact of the response portions 8 with the liquid specimen 4. More specifically, the remover means 32 is a scraping remover means which operates to scrape the unwanted liquid specimen 4 from the response portions 8 after the contact of the response portions 8 with the liquid specimen 4. The scraper remover means 32 comprises a slot 34 for receiving the response portion 8 as the response portions 8 are pushed into and pulled out of the collection container 12. Part of the slot 34 is defined by a wall 36 which scrapes against part of the response portions 8 to remove the unwanted liquid specimen 4.

In a modification of the invention (not shown) the scraper remover means 34 may be located within a recess, thereby preventing an operator being able to make contact with residue liquid specimen 4 remaining around the slot 34. A recessed scraper remover means may be able to provide optimum reduction of the possibility of contamination on the outside of the valve means 18. Alternatively, the scraper remover means 32 may be located inside the collection container 12. Alternatively, a sponge remover means may be employed to remove excess urine.

At least a part of the test container 6 may be transparent in order to allow reading of the response portions 8 in the test container 6 after the response portions 8 have been moved into contact with the liquid specimen 4 and then moved back into the test container 6. The test container may thus be provided with a viewing window (not shown). The viewing window may be in the form of a strip or alternatively the viewing window may be of any other required shape.

The apparatus 2 of the present invention is simple to operate and inexpensive to manufacture. It is able to minimise exposure of an operator such for example as a doctor or a nurse to the liquid specimen 4. In addition, the apparatus 2 is able to be operated such that unwanted excess soaking of the response portions 8 by the liquid specimen 4 is avoided. This unwanted soaking may result in the response portions 8 becoming excessively saturated such that their performance is adversely affected, for example by excess liquid specimen preventing air reaching the response portions for a required chemical change, or for example because the excess response liquid specimen acts to transfer colours from one response portion to another. Thus the performance may be adversely affected such that inaccurate results are given. Still further, in avoiding excess liquid specimen 4 in the test container 6, the problem of excess liquid specimen possibly obscuring viewing of the response portions 8 through a viewing window in the test container 6 is able to be avoided. Because the response portions 8 are clearly able to be seen through the test container 6, then they can be accurately viewed or otherwise read without accuracy possibly being compromised. The response portions 8 may be read by human eye or by digital means, for example digital lens means or digital camera means.

It is to be appreciated that the embodiment of the invention described above with reference to the accompanying drawings has been given by way of example only and that modifications may be effected. Thus, for example, the apparatus 2 may be used without the collection container 12. When the apparatus 2 is used with the collection container 12, then the mover means 10 may move the response portions 8 into and out of the collection container 12 through a side of the body 14 rather than through the lid 16. The collection container 12 may alternatively be a bedpan, with or without a lid. The mover means 10 may be other than a linear acting mover means 10 and thus it may be, for example, a rotary acting mover means. The mover means 10 may be configured to cause the required to and fro movement of the response portion 8 without the response portions 8 being connected to the mover means 10. The remover means 32 may be other than that shown and thus, for example, the remover means 32 may be a squeezing remover means. The liquid specimen 4 shown in the drawings is urine but it may be another type of liquid specimen such for example as blood. Individual components shown in the drawings are not limited to use in their drawings and they may be used in other drawings and in all aspects of the invention. The invention also extends to the individual components mentioned and/or shown above, taken singly or in any combination.

1-16. (canceled)

17. Apparatus for testing a liquid specimen, which apparatus comprises:
(i) a test container;
(ii) a plurality of response portions which are positioned in the test container and which enable the testing of the liquid specimen consequent upon contact with the liquid specimen; and
(iii) mover means for moving the response portions out of the test container for contact with the liquid specimen, and for moving the response portions back into the test container after the contact with the liquid specimen and for the testing of the liquid specimen.

18. Apparatus according to claim 17, in which the contact with the liquid specimen comprises dipping the response portions into the liquid specimen, and in which the moving of the response portions back into the test container comprises removing the response portions from the liquid specimen.

19. Apparatus according to claim 17 and including a collection container for the liquid specimen, and in which the mover means moves the response portions into the collection container for the contact with the liquid specimen, and in which the mover means moves the response portions out of the collection container for the moving of the response portions back into the test container.

20. Apparatus according to claim 19, in which the collection container comprises a body and a lid, and in which the mover means moves the response portions into and out of the collection container through the lid.

21. Apparatus according to claim 19, in which the response portions move into and out of the collection container through the valve means.

22. Apparatus according to claim 21, in which the valve means comprises a valve orifice, and in which the test container opens the valve orifice such that the response portions do not touch the valve means.

23. Apparatus according to claim 17, in which the response portions are connected to the mover means.

24. Apparatus according to claim 17, in which the mover means is a linear acting mover means.

25. Apparatus according to claim 24, in which the linear acting mover means is a plunger linear acting mover means.

26. Apparatus according to claim 25, in which the plunger linear acting mover means comprises an elongate body and a plunger for moving to and fro in the elongate body, and in which the response portions are connected to the plunger.

27. Apparatus according to claim 26, in which the response portions extend longitudinally.

28. Apparatus according to claim 17 and including remover means for removing unwanted liquid specimen from the response portions after the contact of the response portions with the liquid specimen.

29. Apparatus according to claim 28, in which the remover means is a scraping remover means which operates to scrape the unwanted liquid specimen from the response portions after the contact of the response portions with the liquid specimen.

30. Apparatus according to claim 29, in which the scraping remover means comprises a slot for receiving the response portions, and in which at least a part of the slot is defined by a wall which scrapes against at least a part of the response portions.

31. Apparatus according to claim 28, in which the remover means is a sponge remover means.

32. Apparatus according to claim 17, in which at least a part of the test container is transparent in order to allow reading of the response portions in the test container after the response portions have been moved into contact with the liquid specimen and moved back into the test container.

33. Apparatus for testing a liquid specimen, the apparatus comprises a test container, the test container comprises an elongate body and a linear acting plunger movable to and from in the elongate body, a plurality of response portions are provided for testing a liquid specimen consequent upon contact with the liquid specimen, the response portions are carried on or by the linear acting plunger, the plunger is operable to move the response portions out of the body for dipping the response portions into the liquid specimen, the plunger is also operable to remove the response portions from the liquid specimen and to move the response portions back into the body after the contact with the liquid specimen such that unwanted excess soaking of the response portions by the liquid specimen is avoided.

34. Apparatus according to claim 33, in which the response portions can be seen through the body.

35. Apparatus according to claim 33, further comprising a collection container for receiving a liquid specimen.

36. Apparatus according to claim 35, in which the collection container comprises a body and a lid and the plunger moves the response portions into and out of the collection container body through the lid.
